(19) United States
(12) Patent Application Publication
(10) Pub. No.: US 2003/0187504 A1
(43) Pub. Date: Oct. 2, 2003

(54) Adjustable intraocular lens

(76) Inventors: Joseph I. Weinschenk III, Fort Worth, TX (US); Xiaoxiao Zhang, Fort Worth, TX (US)

Correspondence Address: ALCON RESEARCH, LTD., R&D COUNSEL, Q-148, 6201 SOUTH FREEWAY, FORT WORTH, TX, 76134-2099, US

(21) Appl. No.: 10/113,193

(22) Filed: Apr. 1, 2002

(51) Int. Cl. A61F 2/16

(52) U.S. Cl. 623/6.22; 623/6.34; 623/6.46; 623/6.59

(57) ABSTRACT

An adjustable lens system. In a first embodiment, the lens system of the present invention is a two optic system. The optics are connected by an expandable material that allows the distance between the optics to increased in-situ. In a second embodiment of the present invention is a single optic system having a section made from an expandable material at or near the junction between the optic and the centering haptics. Expansion of these section causes the haptics to move radially away from the optic. Such movement may allow for recentering of the lens or, if the haptics are slightly vaulted, radial movement of the haptics away from the optic will cause axial movement of the lens system along the visual axis.

### BACKGROUND OF THE INVENTION

This invention relates generally to the field of intraocular lenses (IOL) and, more particularly, to adjustable IOLs.

The human eye in its simplest terms functions to provide vision by transmitting light through a clear outer portion called the cornea, and focusing the image by way of a crystalline lens onto a retina. The quality of the focused image depends on many factors including the size and shape of the eye, and the transparency of the cornea and the lens.

When age or disease causes the lens to become less transparent, vision deteriorates because of the diminished light which can be transmitted to the retina. This deficiency in the lens of the eye is medically known as a cataract. An accepted treatment for this condition is surgical removal of the lens and replacement of the lens function by an artificial intraocular lens (IOL).

In the United States, the majority of cataractous lenses are removed by a surgical technique called phacoemulsification. During this procedure, an opening is made in the anterior capsule and a thin phacoemulsification cutting tip is inserted into the diseased lens and vibrated ultrasonically. The vibrating cutting tip liquifies or emulsifies the lens so that the lens may be aspirated out of the eye. The diseased lens, once removed, is replaced by an artificial lens.

In the natural lens, bifocality of distance and near vision is provided by a mechanism known as accommodation. The natural lens, early in life, is soft and contained within the capsular bag. The bag is suspended from the ciliary muscle by the zonules. Relaxation of the ciliary muscle tightens the zonules, and stretches the capsular bag. As a result, the natural lens tends to flatten. Tightening of the ciliary muscle relaxes the tension on the zonules, allowing the capsular bag and the natural lens to assume a more rounded shape. In this way, the natural lens can focus alternatively on near and far objects.

As the lens ages, it becomes harder and is less able to change shape in response to the tightening of the ciliary muscle. This makes it harder for the lens to focus on near objects, a medical condition known as presbyopia. Presbyopia affects nearly all adults over the age of 45 or 50.

Prior to the present invention, when a cataract or other disease required the removal of the natural lens and replacement with an artificial IOL, the IOL was a monofocal lens. Most IOLs are sold in power increments of +/−0.5 diopters, and the ultimate power of the lens depends upon where the lens sits along the optical axis. The fixed increment of the lens, and the slight variation in lens placement can result in less than optimum vision. Although this situation occurs relatively infrequently, and generally is not severe, some patients ultimately are required to use a pair of spectacles or contact lenses for optimum vision.

There have been several prior suggested adjustable power IOLs, none of which have been commercially introduced. For example, U.S. Pat. Nos. 5,222,981 (Werblin) and 5,358,520 (Patel), the entire contents of which being incorporated herein by reference, suggest the use of a second or even a third optic that may be implanted and attached to a previously implanted primary optic so as to adjust the overall optic power of the multi-lens system. U.S. Pat. Nos. 5,628,798 and 5,800,533 (Eggleston, et al.), the entire contents of which being incorporated herein by reference, disclose a threadedly adjustable IOL wherein the location of the optic along the visual axis may be adjusted. U.S. Pat. No. 4,575,373 (Johnson), the entire contents of which being incorporated herein by reference, discloses an IOL having an optic and an outer ring and connections between the optic and the outer ring made from a heat-shrinkable plastic. The connections are heated with a laser to adjust the power of the IOL. U.S. Pat. Nos. 4,919,151 and 5,026,783 (Grubbs, et al.), the entire contents of which being incorporated herein by reference, disclose a lens made from a polymer that swells or otherwise changes shape. The lens is implanted or injected into the capsule bag and selectively polymerized so as to adjust the power of the optic. U.S. Pat. No. 5,571,177 (Deacon, et al.), the entire contents of which being incorporated herein by reference, discloses an IOL having haptics with frangible stiffeners. Once implanted in an eye, the stiffeners are selectively cut or heated above their t_{g} by laser radiation, causing the stiffness of the haptic to change and adjusting the location of the lens within the capsule bag. The multi-lens designs and the threadedly adjustable designs all require a secondary surgical procedure in order to make any necessary adjustment to the lens. The adjustment of the lens power by in-situ polymerization of the lens material requires the implantation of a lens made from an unpolymerized, possible toxic material.

Therefore, a need continues to exist for a safe and stable accommodative intraocular lens system that provides adjustment over a broad and useful range.

### BRIEF DESCRIPTION OF THE INVENTION

The present invention improves upon the prior art by providing an adjustable lens system. In a first embodiment, the lens system of the present invention is a two optic system. The optics are connected by an expandable material that allows the distance between the optics to increased in-situ. In a second embodiment of the present invention is a single optic system having a section made from an expandable material at or near the junction between the optic and the centering haptics. Expansion of these section causes the haptics to move radially away from the optic. Such movement may allow for recentering of the lens or, if the haptics are slightly vaulted, radial movement of the haptics away from the optic will cause axial movement of the lens system along the visual axis.

Accordingly, one objective of the present invention is to provide a safe and biocompatible intraocular lens.

Another objective of the present invention is to provide a safe and biocompatible intraocular lens that is easily implanted in the posterior chamber.

Still another objective of the present invention is to provide a safe and biocompatible intraocular lens that is stable in the posterior chamber.

Still another objective of the present invention is to provide a safe and biocompatible adjustable lens system.

These and other advantages and objectives of the present invention will become apparent from the detailed description and claims that follow.

### BRIEF DESCRIPTION OF THE DRAWING

FIGS. 1A-1B are enlarged partial cross-sectional views of a first embodiment of the lens system of the present system.

FIGS. 2A-2B are perspective views of a second embodiment of the lens system of the present invention.

FIGS. 3A-3B are plan views of a third embodiment of the lens system of the present invention.

### DETAILED DESCRIPTION OF THE INVENTION

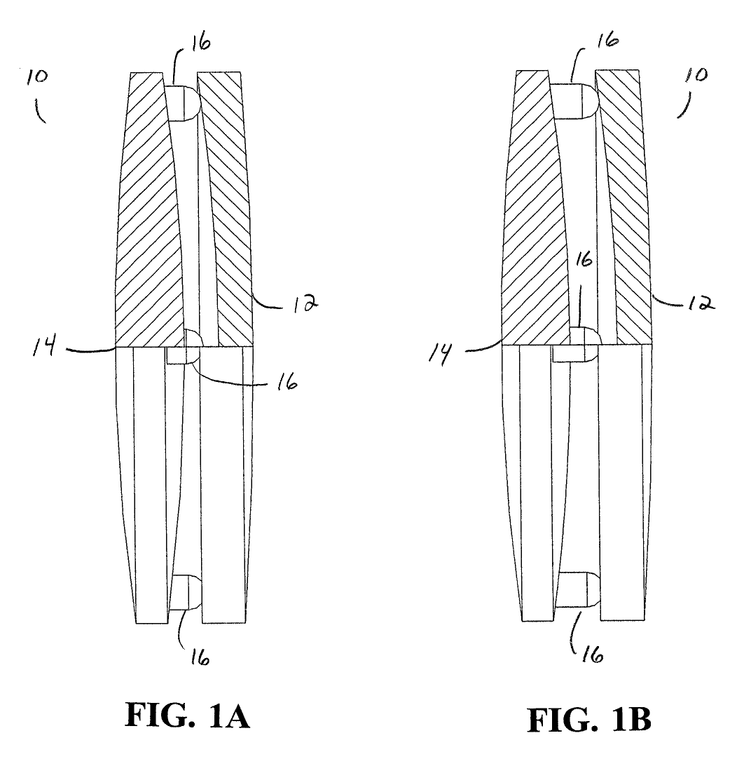

As best seen in FIGS. 1A-1B, lens system 10 of the present invention generally consists of posterior optic 12 and anterior optic 14. Optics 12 and 14 are preferably formed in any suitable overall diameter, for example, between approximately 4.5 millimeters and 6.5 millimeters and made from a soft, foldable material such as a hydrogel, silicone or a soft acrylic. Optics 12 and 14 may be any powers suitable to satisfy the overall power requirements of lens system 10. The relative powers of optics 12 and 14 should be such that the radial movement of optic 14 toward or away from optic 12 should be sufficient to adjust the overall power of lens system 10 at least 0.1 diopters and preferably, at least from about 0.1 diopters to about 5.0 diopters, calculation of such powers of optics 12 and 14 being within the capabilities of one skilled in the art of designing ophthalmic lenses by, for example, using the following equations:

P=P₁+P₂−T/n*P₁P₂ (1)

δP=δT/n*P₁P₂ (2)

Optics 12 and 14 are connected by a series of protuberances or columns 16. Columns 16 are made from an expansive material. Expanding columns 16 in the manner discussed below forces optics 12 and 14 apart from each other, thereby changing the power of lens system 10. In addition, selective expansion of columns 16 will cause selective portions of optic 12 away from optic 14, thereby adjusting the power of lens system 10 to account for any astigmative error in the eye.

By way of example, columns 16 can comprise a masked hydrogel material. Exposure of this material to a suitable unmasking agent would enable the material to absorb water, resulting in the material's expansion. The masking could be accomplished by a hydrophobic material coating that could be non-toxically degraded by exposure to laser energy. Alternatively, columns 16 could can a material capable of being converted to material of higher water content. The material could be initially hydrophobic or hydrophilic. As an example, the material could contain anhydride chemical moieties that would undergo scission when exposed to the appropriate thermal or electromagnetic radiation. In the presence of trace amounts of water, this would convert each anhydride moiety to two carboxylate moieties. Since carboxylates are very hydrophilic chemical structures, the resulting chemically altered expansile zone would be more hydrophilic.

This would lead to its absorption of a significant amount of water.

Alternatively, columns 16 can comprise certain monomers known to occupy less volume in their pre-polymerization state than in their polymerized state, for example, spiro ortho carbonates. To use such monomers, these monomers need to be encapsulated in an elastic material. Therefore, columns 16, before activation, will be a reservoir containing expandable monomer. Columns 16 are activated by exposure to appropriate thermal or electromagnetic radiation. This energy exposure would cause polymerization within columns 16 and as the polymerization proceeded, columns 16 will increase in size.

Additionally, columns 16 can comprise a material not in its natural resting state. In other words, columns 16 can initially be in an unstressed state that resulted in optics 12 and 14 being farther apart than actually desired. Lens system 10 then undergoes suitable processing such that columns 16 were compressed, and the compression “locked in” until a relieving force was applied. As an example, columns 16 can comprise a cross-linked copolymer of 2-phenylethyl acrylate and 2-phenylethyl methacrylate. This copolymer would have an appropriate composition so that its glass transition would be about 45° C. At room temperature (about 24° C.) columns 16 will be relatively rigid. Warming of columns 16 above 45° C., will cause columns 16 to become relatively rubbery and deformable. Optics 12 and 14 are compressed toward each other (compressing columns 16) and lens system 10 cooled to room temperature. When cooled, columns 16 will remain in their compressed state because their temperature is well below their glass transition temperature. Columns 16 can then be expanded by using focused laser light that would heat columns 16 above 45° C., and the amount of expansion can be controlled by the duration and intensity of the heating. For example, very limited heating time might cause only 10% of the total expansion possible.

Alternatively, columns 16 can comprise a material that undergoes a shape change to due a change in its environment, such as a pH or hydration increase or decrease. For example, certain acrylamide polymers are known to be highly sensitive to pH and/or water content, and undergo significant shape changes (contraction or elongation) when their environment is altered. This material behavior could be exploited, for example, by constructing columns 16 of a mixture of a shape-changeable acrylamide polymer, and a polymer with chemical functionalities that could be modified, so that the overall polymeric mixture could be made to absorb more water or undergo a pH change. As described above, a polymer with anhydride moieties can be altered to become a more hydrophilic polymer. Therefore, the composition used to form columns 16 will be part acrylamide polymer and part anhydride-containing polymer. When on lens system 10, columns 16 can be caused to expand by scission of its anhydride moieties, which would cause increased water content, thereby causing the appropriately formulated polyacrylamide to elongate.

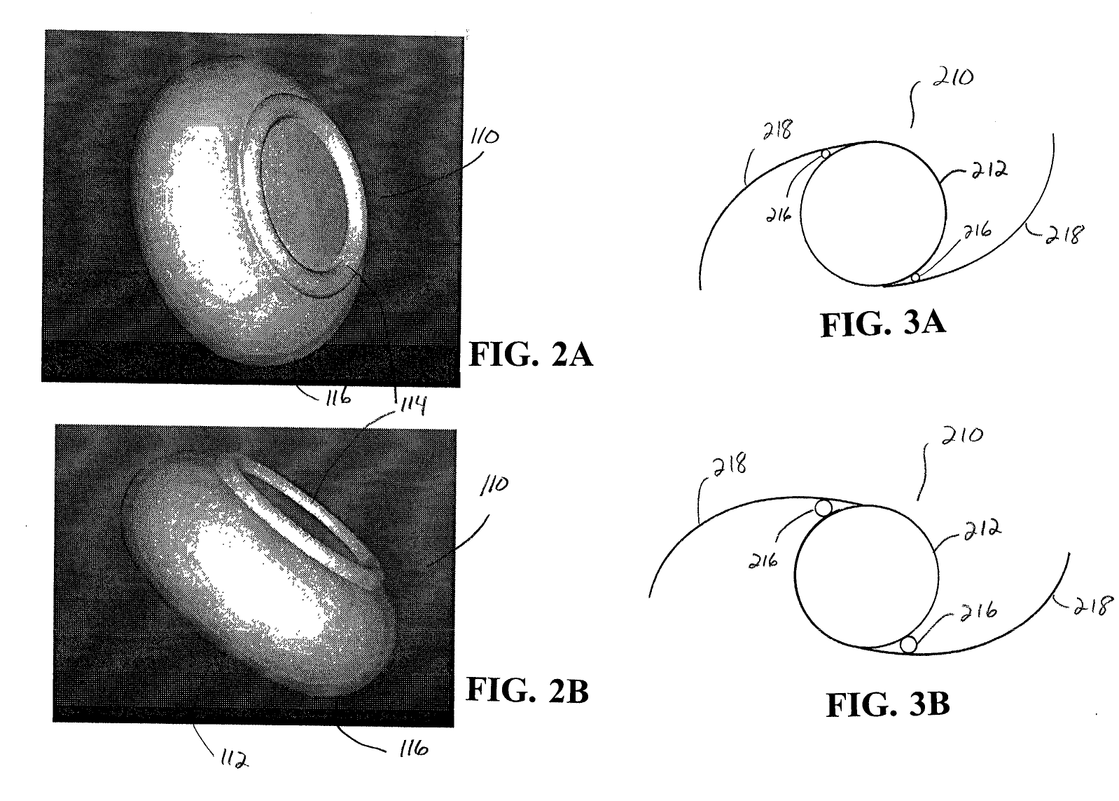

As best seen in FIGS. 2A-2B, lens system 110 may contain anterior optic 114 and posterior optic 112 separated by expansive bladder 116. Bladder 116 can be expanded in much the same manner as columns 16 to vary the spacing between anterior optic 114 and posterior optic 112.

As best seen in FIGS. 3A-3B lens 210 of another embodiment of the present invention may contain single optic 212 having at least a pair of haptics 218. Optic 212 is preferably formed in any suitable overall diameter, for example, between approximately 4.5 millimeters and 6.5 millimeters and made from a soft, foldable material such as a hydrogel, silicone or a soft acrylic. Optic 12 may be any power suitable to satisfy the overall power requirements of lens 210. Haptics 218 may be integrally formed with optic 212 or may be formed separately of any suitable thermoplastic and attached to optic 112 in any conventional manner, such haptic attachment methods being well-known in the art. At or near the attachment points of haptics 218 and optic 212 are buttons 216 made from a material similar to those discussed above with respect to columns 16. As seen in FIG. 3A, lens 210 is implanted with buttons 216 in an unexpanded state. As seen in FIG. 3B, following implantation, if needed, buttons 216 can be expanded in the manner discussed above, thereby forcing haptics 218 away from optic 212, thereby lengthening lens 210. Such lengthening of lens 210 will adjust the position of optic 212 along the visual axis, particularly if haptics 218 are vaulted (attached to optic 210 at an angle, for example between approximately 0° and 10°).

This description is given for purposes of illustration and explanation. It will be apparent to those skilled in the relevant art that changes and modifications may be made to the invention described above without departing from its scope or spirit.

We claim:

1. An intraocular lens system, comprising:
a) a first optic;
b) a second optic; and
c) at least one column joining the first optic to the second optic, the column being made from an expansive material.

2. The lens system of claim 1 wherein the expansive material is a masked hydrogel.

3. The lens system of claim 1 wherein the expansive material comprises a monomer known to occupy less volume in its pre-polymerization state than in its polymerized state.

4. The lens system of claim 1 wherein the expansive material is an acrylamide polymer.

5. The lens system of claim 1 wherein the expansive material is a cross-linked copolymer of 2-phenylethyl acrylate and 2-phenylethyl methacrylate.

6. The lens system of claim 1 wherein the expansive material comprises an acrylamide polymer and an anhydride-containing polymer.

7. The lens system of claim 6 wherein the expansive material is caused to expand by scission of its anhydride moieties.

8. The lens system of claim 1 wherein the columns cause the second optic to vault away from the first optic.

9. The lens system of claim 1 wherein the first optic and the second optic comprise a soft acrylic.

10. The lens system of claim 1 wherein the second optic comprises a hydrogel.

11. The lens system of claim 1 wherein the second optic comprises silicone.

12. The lens system of claim 1 wherein the first optic comprises silicone.

13. The lens system of claim 1 wherein the first optic comprises a hydrogel.

14. An intraocular lens, comprising:
a) an optic;
b) at least one pair of haptics attached to the optic at least a pair of junctions; and
c) a button located at each of the junctions of the haptic with the optic, the button being made from an expansive material.

15. The lens of claim 14 wherein the expansive material is a masked hydrogel.

16. The lens of claim 14 wherein the expansive material comprises a monomer known to occupy less volume in its pre-polymerization state than in its polymerized state.

17. The lens of claim 14 wherein the expansive material is an acrylamide polymer.

18. The lens of claim 14 wherein the expansive material is a cross-linked copolymer of 2-phenylethyl acrylate and 2-phenylethyl methacrylate.

19. The lens of claim 14 wherein the expansive material comprises an acrylamide polymer and an anhydride-containing polymer.

20. The lens of claim 19 wherein the expansive material is caused to expand by scission of its anhydride moieties.

21. The lens of claim 14 wherein expansion of the button causes the haptics to be pushed away from the optic.

22. The lens of claim 14 wherein the optic comprises a soft acrylic.

23. The lens of claim 14 wherein the optic comprises a hydrogel.

24. The lens of claim 14 wherein the optic comprises silicone.

25. An intraocular lens system, comprising:
a) a first optic;
b) a second optic; and
c) a bladder joining the first optic to the second optic, the bladder being made from an expansive material.

26. The lens system of claim 25 wherein the expansive material is a masked hydrogel.

27. The lens system of claim 25 wherein the expansive material comprises a monomer known to occupy less volume in its pre-polymerization state than in its polymerized state.

28. The lens system of claim 25 wherein the expansive material is an acrylamide polymer.

29. The lens system of claim 25 wherein the expansive material is a cross-linked copolymer of 2-phenylethyl acrylate and 2-phenylethyl methacrylate.

30. The lens system of claim 25 wherein the expansive material comprises an acrylamide polymer and an anhydride-containing polymer.

31. The lens system of claim 30 wherein the expansive material is caused to expand by scission of its anhydride moieties.

32. The lens system of claim 25 wherein the columns cause the second optic to vault away from the first optic.

33. The lens system of claim 25 wherein the first optic and the second optic comprise a soft acrylic.

34. The lens system of claim 25 wherein the second optic comprises a hydrogel.

35. The lens system of claim 25 wherein the second optic comprises silicone.

36. The lens system of claim 25 wherein the first optic comprises silicone.

37. The lens system of claim 25 wherein the first optic comprises a hydrogel.

38. The lens system of claim 1 wherein expanding the column adjusts the power of the lens system to account for an astigmative error.

39. The lens system of claim 25 wherein expanding the bladder adjusts the lens system to account for an astigmative error.
